(19) United States
(12) Patent Application Publication
(10) Pub. No.: US 2012/0203192 A1
(43) Pub. Date: Aug. 9, 2012

(54) Ashesive Transfer Layer for Use in a Body Adhering Absorbent Article

(76) Inventors: Melissa Jean Dennis, Appleton, WI (US); Perry Allen Bush, Menasha, WI (US); Donald James Schmidt, Neenah, WI (US); Todd William Wilkes, Appleton, WI (US); David John Enz, Neenah, WI (US); John Timothy Hahn, Merrill, WI (US)

(21) Appl. No.: 13/022,697

(22) Filed: Feb. 8, 2011

(51) Int. Cl. A61F 13/60; A61F 13/15; C09J 5/02; B32B 37/20; B32B 38/14

(52) U.S. Cl. 604/385.05; 156/324.4; 156/275.5; 156/277; 156/204

(57) ABSTRACT

Generally, the present disclosure relates to an adhesive transfer layer that may be incorporated in a body adhering absorbent article. Various adhesive transfer layers and methods of constructing the same are described. For example, in one embodiment the adhesive transfer layer (ATL) may comprise a construction adhesive, a body attachment adhesive, a carrier and a peel strip.

### FIELD OF THE INVENTION

This disclosure relates to methods of making an adhesive transfer layer (ATL) for use in a body adhering absorbent article and methods of manufacturing the same. Various adhesive transfer layers are disclosed as well as methods of constructing the same and incorporating them into individual body adhering absorbent articles.

### BACKGROUND

Disposable absorbent articles for the absorption and containment of urine, menses and other body exudates are generally known in the art. Generally, these articles are referred to as absorbent personal care articles and have taken various forms including, diapers for infants and children, training pants for children, sanitary napkins, pantiliners, incontinence pads, incontinence garments and the like, for teenagers and adults. Of these absorbent personal care articles, sanitary napkins, incontinence pads and pantiliners are articles which are not garment-like in form, meaning that they do not rely on the body structure of the user to stay in place during use. For example, diapers use the hips and waist of the user to hold the diaper in place during use. As a result, sanitary napkins, pantiliners and incontinence pads are less noticeable and more discrete than diapers and incontinence garments.

Instead, sanitary napkins, pantiliners, and incontinence pads use an undergarment attaching device to hold the article in place during use. Conventional means of holding these absorbent personal care articles in place include, for example, garment attachment adhesives, which are generally pressure sensitive adhesives; attachment panels, which are sometimes referred to as wings or flaps, extending from the longitudinal side edges of the absorbent article which wrap around the undergarment of the user; or a body adhesive, which secures the absorbent personal care article directly to the body of a user. In addition, combinations of the attachment devices have also been used.

In selecting an attachment device, several considerations must be considered and balanced. The attachment device must hold the absorbent personal care article in place, providing adequate protection, the attachment device must be comfortable, and the attachment device must be convenient and easy to use. Currently, the most common attachment device is a garment adhesive. While absorbent personal care articles with the garment adhesive attachment device have performed well, remaining in place and providing the user with ease of placement and removal, these absorbent personal care articles have suffered from certain drawbacks. For example, the inner crotch surface of the undergarment to which the absorbent personal care articles are adhered is constantly being distorted, twisted and stretched due to the movements of the wearer. As a result, frequently the garment adhesive detaches with the undesirable result of the sanitary napkin, pantiliner or incontinence pad moving out of position. In an extreme case, detachment of the adhesive may also result in the adhesive folding over on itself and then becoming unavailable for reattachment to the undergarment of the user.

In the case of sanitary napkins, some sanitary napkins have been provided with attachment panels which, in use, are generally folded around the crotch portion of the undergarment and affixed to the outer crotch portion. Although such attachment panels have been partially successful in protecting certain regions of the wearer's undergarment, such sanitary napkins are still subject to the forces which cause the sanitary napkin to be distorted, twisted or stretched.

There is a need in the art for an absorbent personal care article, in the form of a sanitary napkin, incontinence pad or pantiliner, which can be effectively held into place against a user's body, providing an effective means to hold the absorbent personal care article in place, thereby providing adequate protection to the user, providing comfort to the user and providing an attachment device which is convenient and easy to use.

Accordingly, the present disclosure provides an ATL which may be used to supply a body adhesive on the body facing surface of an absorbent article to secure the absorbent article to the wearer's body.

### SUMMARY

It has now been discovered that a body adhering absorbent article may be provided with an improved ATL having a removable peel strip which covers the body attachment means of a body adhering absorbent article. In a preferred embodiment the body attachment means is discontinuous relative to the peel strip which covers it providing a user grasping section for easy removal of the peel strip. Accordingly, in one embodiment the present disclosure provides an ATL comprising a construction adhesive, a body attachment adhesive, a carrier and a peel strip.

In still other embodiments the present disclosure provides a method of manufacturing an ATL comprising the steps of providing a web of nonwoven carrier; providing a web of peel strip; applying a body attachment adhesive to the peel strip; and attaching the nonwoven carrier.

In yet other embodiments the present disclosure provides a method of manufacturing an ATL comprising the steps of providing a lower web of peel strip and a web of nonwoven carrier; applying construction adhesive to the nonwoven carrier; attaching the nonwoven carrier to the lower web of peel strip; supplying an upper web of peel strip; applying body attachment adhesive to the upper web of peel strip; and attaching the nonwoven carrier and the upper web of peel strip.

### DESCRIPTION OF THE DRAWINGS

FIG. 1 is a perspective view of a body adhering absorbent article according to one embodiment of the present disclosure;

FIG. 2 is a cross sectional view of an ATL according to one embodiment of the present disclosure;

FIG. 3 is a cross sectional view of an ATL according to another embodiment of the present disclosure;

FIG. 4 is a detailed view of the peel strip according to one embodiment of the present disclosure;

FIG. 5 is a perspective view of a body adhering absorbent article according to another embodiment of the present disclosure;

FIG. 6 illustrates one embodiment for manufacturing an ATL according to the present disclosure;

FIG. 7 illustrates another embodiment for manufacturing an ATL according to the present disclosure;

FIG. 8 illustrates still another embodiment for manufacturing an ATL according to the present disclosure; and

FIG. 9 illustrates yet another embodiment for manufacturing an ATL according to the present disclosure.

### DEFINITIONS

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

“Absorbent structure” refers to the central fluid handling portion of an absorbent article and may include but is not limited to one or more of the following components: cellulose fluff, superabsorbent material, coform, absorbent sponge material, surge material, or wicking material but does not include a topsheet or backsheet.

“Absorbent component” may include but is not limited to one or more of the following components: an absorbent structure, as defined above, a fluid pervious topsheet, a fluid impervious backsheet, or attachment adhesive.

As used herein, the term “attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or a refastenable attachment. In addition, the attachment can be completed either during the manufacturing process or by the end wearer.

As used herein, the term “body-facing” means that surface or side of the article which is intended to be disposed toward or placed adjacent to the body of the wearer during ordinary use. The term “garment-facing” means that surface or side that is on the opposite side of the article from the body-facing surface or side. The garment-facing surface is an outward surface of the article and is intended to be disposed to face away from the wearer's body during ordinary use. The garment-facing surface is generally arranged to face toward or placed adjacent to the wearer's undergarments or clothing when the article is worn.

As used herein, the term “connected” is intended to mean directly connected and indirectly connected. By directly connected, it is intended that the connected elements are in contact with one another or affixed to one another. By indirectly connected, it is intended that one or more intervening or intermediate elements are between the two elements which are secured or “connected” together. The intervening elements may be affixed.

### DETAILED DESCRIPTION

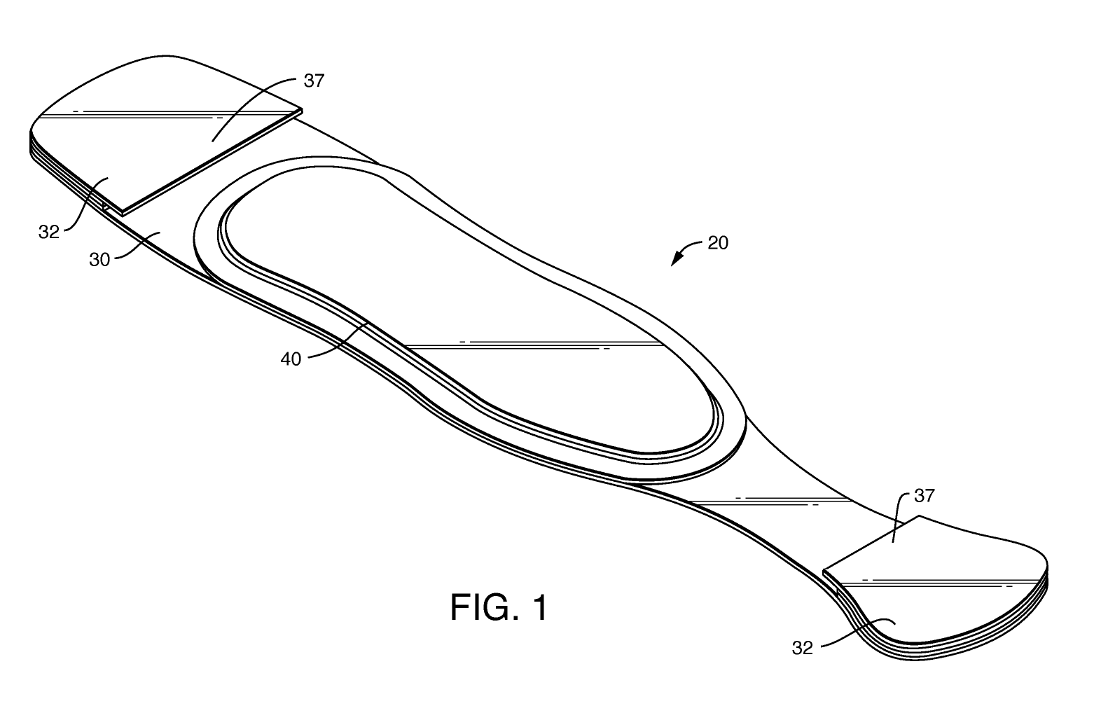

Generally, the present disclosure relates to an ATL that may be incorporated in a body adhering absorbent article. Various adhesive transfer layers and methods of constructing the same are described. Now with reference to FIG. 1, one embodiment of a body adhering absorbent article 20 is illustrated. Body adhering absorbent article 20 comprises a body adhering shell 29, and an absorbent article component 40. The body adhering shell 29 further comprises a body adhesive, for example an adhesive transfer layer (ATL) 32.

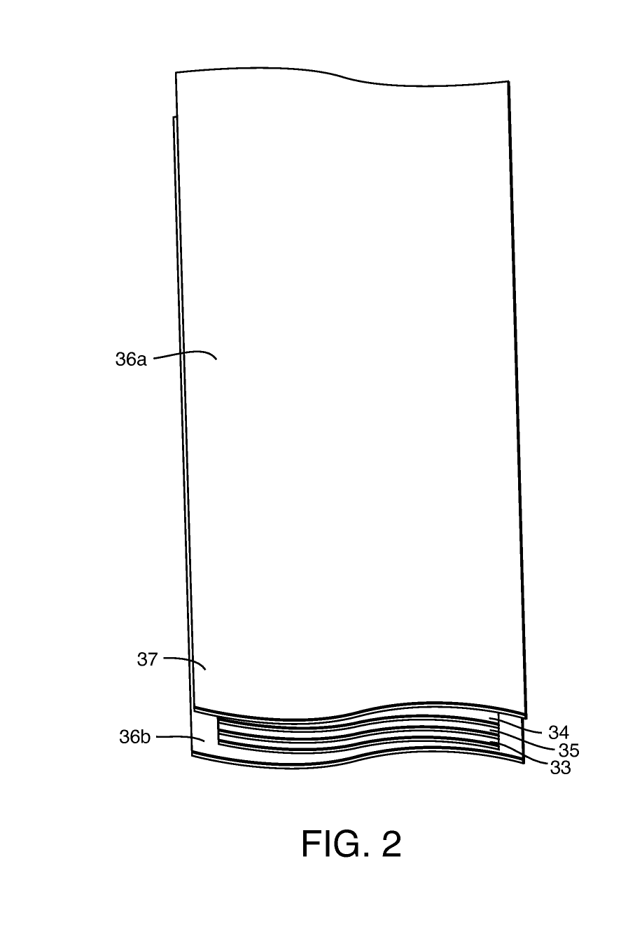

One configuration of the ATL 32 is shown in FIG. 2. In this embodiment the ATL 32 consists of a lower peel strip 36b, a construction adhesive 33, a body attachment adhesive 34, a carrier 35, and an upper peel strip 36a. In this configuration the ATL may be incorporated in a body adhesive absorbent article, as illustrated in FIG. 1, such that in use the user removes the upper peel strip 36a and the exposed body attachment adhesive adheres the absorbent article to the user's body.

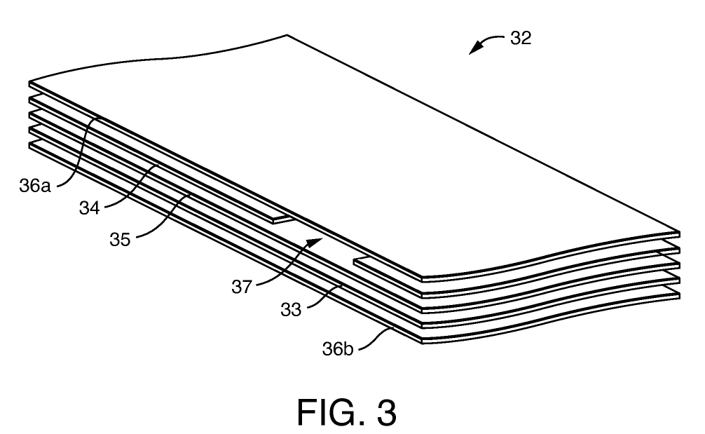

Another configuration of the ATL 32 is shown in FIG. 3. In this embodiment the ATL 32 consists of a lower peel strip 36b, a construction adhesive 33, a body attachment adhesive 34, a carrier 35, an upper peel strip 36a and a finger tab 37. In this configuration, the finger tab 37 allows the user to grasp the upper peel strip 36a by placing her thumb under the unattached peel strip region, and grasping the finger tab 37 between the thumb and forefinger the user applies a peeling force to remove the upper peel strip 36a. Other configurations of the finger tab 37, as well as methods of making the same will be discussed in more detail below.

The construction adhesive 33 may be any adhesive known in the art suitable for adhering an elastic material to a non-woven material. Particularly preferred construction adhesives include hot melt adhesives. Preferably the hot melt adhesives are soft and suitable for elastic attachment of elastic material to disposable substrates such as non-woven materials and polyethylene. Examples of appropriate hot melt adhesives are those commonly used in the industry for adhering an elastic material to a non-woven material. Particularly preferred examples of suitable adhesives include, EASYMELT™ (Nation Starch Chemical Co., USA), HL-8130-E 2P (H.B Fuller Company, US) and Sanicare SH4-187 HV™ (Henkel, USA). Other commercially available construction adhesives usable in the present invention include, for example Rextac adhesives available from Huntsman Polymers of Houston, Tex., as well as adhesives available from Bostik Findley, Inc, of Wauwatosa, Wis. A particularly preferred construction adhesive is EASYMELT™ 34-5610.

As stated above, the body attachment adhesive 34 enables the attachment of the absorbent article to a wearer. Stated another way, the ATL 32 is the body attachment member portion of the article that is attached to the body of the wearer. Preferably the body attachment adhesive 34 contacts the skin and hair, if present, in the vulva region and possibly the pubic region and/or the perinea region of the wearer's body, thereby supporting and holding the absorbent article 40 against the body of the wearer during use. The body adhesive 34 can overlie a portion of the first side of the carrier 35 or it may overlie the entirety of the carrier 35. Generally, the body adhesive 34 will be present on at least the outer portion first side of the carrier 35 such that it is positioned near the edge of the absorbent article 40. The body adhesive 34 may also be placed in a pattern of the carrier 35. The body adhesive 34 can be applied to the first side of the carrier 35 using any known process including inkjet printing, screen printing, or extruding the body adhesive 34 from one or more nozzles, slot coating and the like.

Generally, any pressure sensitive adhesive known to those skilled in the art may be used for the body adhesive 34, provided that the pressure sensitive adhesive is not a known irritant to human skin or that the adhesive is so aggressive that it causes pain to the wearer when the absorbent article is removed from the skin. It is also desirable that the adhesive is selected such that the adhesive does not leave a substantial amount of residue on the skin of the wearer, when the absorbent article 40 is removed. Particularly suitable pressure sensitive adhesive materials are disclosed in U.S. Pat. Nos. 6,213,993 and 6,620,143, both of which are incorporated herein in a manner consistent with the present disclosure. Other suitable adhesives are disclosed in U.S. Pat. Nos. 5,618,281 and 6,316,524, both of which are incorporated herein in a manner consistent with the present disclosure. Additional examples of pressure sensitive adhesives include hydrogels, hydrocolloids, acrylics based adhesives, and rubber based adhesives, such as Kraton based adhesives.

In certain embodiments the body attachment adhesive preferably comprises a UV curable polymer. As used herein, the term “UV curable polymer,” generally refers to one or more polymers comprising a photoinitiator that is covalently bound to the polymer. Preferred are polymers comprising an acrylic polymer backbone molecule that is modified with polymerized photoreactive groups, e.g., a modified benzophenone group that is chemically bonded to the acrylic polymer chain. The polymer is crosslinked by chemical grafting caused by the excitation of the photo initiator by UV irradiation.

UV curable polymers are commercially available from a number of suppliers and are typically solvent- and water-free acrylic raw material that can be used for the production of pressure sensitive tapes and labels. These polymers are nearly solid material at room temperature and have to be heated to a temperature of about 120-130° C. to become fluid enough for the coating process. At this temperature, they can be applied to the backing substrate or carrier with conventional hot melt coating systems. Thus they are processed as hot melts. After being coated on the carrier, the polymer film is crosslinked by UV-irradiation to produce the adhesive properties required.

The body adhesive 34 may be positioned on the first side of the carrier 35 in an open pattern or a closed pattern. By “open pattern” it is meant that the adhesive can have an intermittent or discontinuous pattern that does not substantially encircle the entire first side of the carrier 35. For example, there may be breaks in the body adhesive at certain portions of the first side of the carrier 35. “Closed pattern” means the adhesive 34 would encircle the entire opening 105 in the shell. In one embodiment, the pattern of the body adhesive 34 will substantially surround the cover of the first side of the carrier 35. An example of an “open” pattern of the adhesive would be to have individual beads of adhesive applied in a discontinuous fashion. The closed pattern can be advantageous since the body adhesive 34 may form a seal with the body of the wearer which will assist in preventing leaks from the absorbent article 100. The body adhesive may form a dam, which may prevent leaks from the entire perimeter of the absorbent article 100.

In one embodiment, the body adhesive 34 may be placed on the entire first side of the carrier 35, as seen in FIG. 2. In another embodiment, the body adhesive 34 may be placed along the outer portions of the first side of the carrier 35 near the periphery. The adhesive 34 may be applied in a pattern of small discrete dots so as to leave numerous areas free from adhesive. Alternatively, the adhesive 34 may be applied as a continuous bead, or may be applied as a series of semi-continuous beads. Other suitable adhesive patterns may be selected for applying the body adhesive 34 to the body-contacting first side of the carrier. For example, adhesive patterns can be oval, swirls, various linear or non-linear arrays of adhesive longitudinally, and/or transversely oriented and reticulated webs having unobstructed interstices between the adhesive fibers or combinations thereof. As stated above, the adhesive patterns may be open or closed. The weights of adhesives are limited to less than about 800 grams per square meter, and generally less than about 400 grams per square meter. Generally, the weight of the adhesive is at least 20 grams per square meter. Typically, the adhesive is applied in an amount of about 100 to about 400 grams per square meter. The limitations on the basis weight of the adhesive are important to provide the correct adhesive characteristics for applying directly to the wearer's vulva region and optionally the pubic and perinea regions of the wearer's body. If the basis weight is too high, the absorbent article will have a sticky feeling or otherwise uncomfortable feeling. If the basis weight of the adhesive is too low, there may be insufficient adhesion to the body of the wearer.

Generally, the body adhesive 34 is applied in a manner that is symmetrical about the longitudinal axis that bisects the ATL 32. This symmetrical pattern provides the wearer a balanced feel when wearing the absorbent article 40. The symmetrical pattern also reduces the perception of any associated discomfort when the absorbent article 40 is removed from the body.

As seen in FIGS. 2 and 3, to protect the body adhesive 34, a peel sheet, release sheet or peel strip 36 may be used to prevent the body adhesive 34 from becoming contaminated, thus losing its ability to stick to the body of a wearer and/or prematurely adhering to an unintended surface. The peel strip 36 may be any release liner or transfer liner known to those skilled in the art. The peel strip 36 preferably is capable of being placed in intimate contact with an adhesive and subsequently removed without damaging the adhesive layer.

Suitable materials for use as the peel strip 36 are well known in the art and are commercially available. Examples of suitable peel strip 36 include, a silicone coated Kraft paper, a silicone coated film, or the like. Other release coatings include coatings containing polytetrafluoroethylene. The peel strip 36 may extend beyond one or both of the ends and/or sides of the shell, as shown in FIG. 1. Alternatively, the release sheet may be sized to only cover the body adhesive on the first side of the shell. In yet another embodiment, the release sheet may extend beyond the adhesive at one or more locations by providing the release sheet with a tab for the wearer to grasp to remove the release sheet from the absorbent article and the body adhesive on the absorbent article. When the release sheet extends beyond the adhesive, it is generally easier for the wearer to remove the release sheet to place the absorbent article for use.

Alternatively, the release sheet may be provided with a pressure sensitive adhesive to hold the release sheet in place when the absorbent article is devoid of an adhesive for body attachment. In this configuration, the release sheet serves to protect the absorbent structure and first side of the shell from dirt and damage prior to use.

In another embodiment, the release sheet may not be necessary. For example, the absorbent article may be rolled, folded onto itself or stacked upon each other. In these configurations, a release sheet is not needed. If rolled, the body adhesive will generally contact the second side of the shell or the liquid impermeable backsheet of the absorbent structure. The body adhesive should releasably stick to one second side of the shell by readily releasing when unrolled by the wearer. In addition, the body adhesive should not leave a residue on the second side of the shell, of the backsheet. This should similarly occur when the absorbent articles are stacked upon each other such that the body adhesive of one article will contact the second side of the shell and/or backing sheet of a second article. In another possible configuration, the absorbent article may be folded along the longitudinal axis of the lateral axis such that the body adhesive in one area comes into contact with body adhesive in another area. In the folded configuration, the body adhesive should be selected such that the body adhesive will release from itself when manipulated by a wearer.

The carrier 35 is preferably a flexible material that may be formed into a continuous web. Suitable carrier materials include nonwovens and papers as well as combinations of the same with cotton fabrics, viscose fabrics, cotton-viscose blended fabrics, synthetic fiber wovens, and synthetic fiber nonwovens. Suitable as synthetic fibers are, in particular, polyesters, polyamides and polyurethane. Particularly well suited carrier materials include nonwoven fabrics such as air laid webs of natural or synthetic fibers or blends thereof. The layer thickness of the carrier is preferably 0.1 to 10 mm, preferably 0.5 to 5 mm.

With further reference to FIG. 1, in addition to the shell material 30 and ATL 32, the body adhering absorbent article 20 may comprise an absorbent article component 40, which is generally attached to the first side of the shell material 30. The attachment may be in a permanent manner, meaning that the absorbent article component 40 is generally intended not to be removable by the wearer of the article 20. Alternatively, the absorbent article component 40 may be constructed to be removable by the wearer, meaning that the absorbent article component 40 may be removed and replaced with another absorbent article component 40 by the wearer of the article 20, or be replaced with nothing at all. In some aspects, when the absorbent article component 40 is attached to the shell material 30 in a permanent manner, meaning that the absorbent article component 40 is not intended to be removed by the wearer, various bonding means can be used, such as a construction adhesive for example. Examples of useable construction adhesives include any adhesive which will effectively hold the absorbent article component 40 in place, so as not to be separated from the shell material 30. Commercially available construction adhesives usable in the present invention include, for example, Rextac™ adhesives available from Huntsman Polymers, Houston, Tex. Other means may be used to hold the absorbent article component 40 to the shell material 30 including bonding techniques known in the art, including, but not limited to, adhesive bonds, cohesive bonds, thermal bonds, ultrasonic bonds, embossing, crimping, entangling, fusing, hook and loop, or the like, and combinations thereof.

Where the absorbent article component 40 is preferably removably attached, the absorbent article component 40 is held in place on the shell material 30 by a means which will allow the wearer to remove the absorbent article component 40. One such means of holding the absorbent article component 40 is by using a pressure sensitive adhesive. Suitable pressure sensitive adhesives include, but are not limited to, any commercially available pressure sensitive adhesive. Examples of suitable pressure sensitive adhesives usable to removably hold the absorbent article 40 in place on the shell material 30 include pressure sensitive adhesives available from National Starch, Bridgewater, N.J.

In certain embodiments it may be advantageous for the absorbent article 40 to have a backsheet 42 and more preferably a fluid impervious backsheet. The backsheet can serve to provide liquid impermeability for the absorbent article component 40, such that any fluids entering the absorbent structure 24 will not flow through the structure to the clothing of a wearer. One example of a commercially available fluid impervious backsheet is the XP-3473a baffle available from Huntsman Packaging Corporation, Houston, Tex.

The absorbent structure may comprise wicking layers 43a and 43b, which may be formed from meltblown microfiber such as the 50 gsm meltblown fibers commercially available from Yuhan-Kimberly Ltd., Korea. The absorbent structure 24 may further comprise an absorbent layer 44, intake layer 45 and topsheet 46. The absorbent layer 44 may contain one or more layers of absorbent materials, such as fibrous materials and/or superabsorbent materials for example. Each of the layers can contain similar materials or different materials. Materials that can be used to form the absorbent layer 44 include those materials conventionally used in absorbent articles and includes materials, such as, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A desired material is wood pulp fluff, for it is low in cost, relatively easy to form, and has good absorbency.

In a preferred embodiment, as illustrated in FIG. 1, the body adhering absorbent article 20 comprises a peel strip 36, which extends in the longitudinal direction (machine direction) 76 beyond the extent of the body attachment adhesive 34 forming a user grasping section also know as a finger tab 37. The presence of finger tab 37 allows the user to grasp the peel strip by placing their thumb under the unattached peel strip region and grasping the peel strip in the user grasping section 37 between the thumb and forefinger. The user of the product applies a peeling force to remove the anterior and posterior peel strips 36.

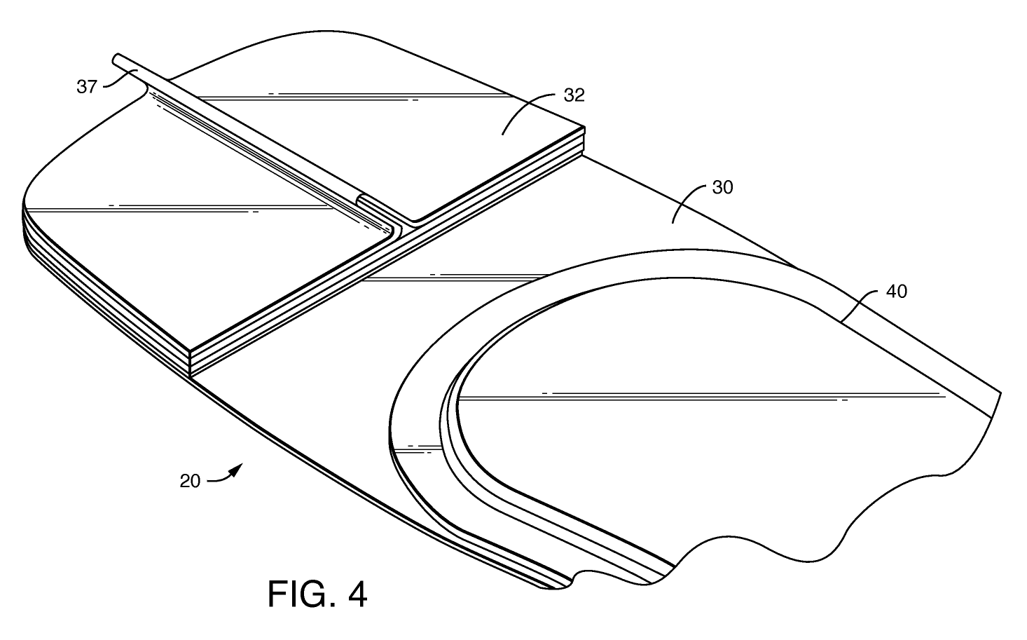

In still another embodiment, illustrated in FIG. 4, the peel strip 36 may be placed over the body attachment adhesive 34 such that a portion of the peel strip 36 forms a ridge extending upward from the adhesive 34. Preferably the ridge extends in the longitudinal (machine) direction and is located approximately in the midpoint of the adhesive 34. The ridge forms a user grasping section that allows the user to grasp the peel strip by placing their thumb and forefinger on the unattached peel strip region. The user of the product applies an upward force to remove the peel strips 36.

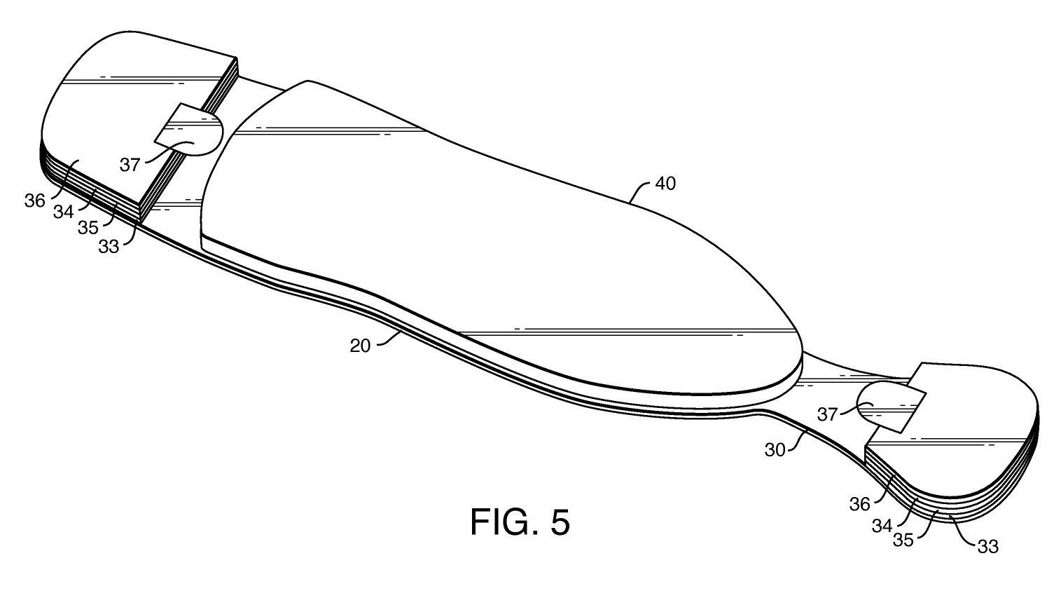

In yet another embodiment, illustrated in FIG. 5, the body adhering absorbent article 20 comprises a peel strip 36, which has a tab bonded to the peel strip 36 forming a user grasping section also know as a finger tab 37. The presence of finger tab 37 allows the user to grasp the peel strip by placing their thumb under the unattached peel strip region and grasping the peel strip in the user grasping section 37 between the thumb and forefinger. The user of the product applies a peeling force to remove the anterior and posterior peel strips 36.

In still other embodiments the peel strip may be applied as two or more discrete pieces such that a seam is formed between adjacent peel strips. In such embodiments the seam formed by two adjacent peel strips has a pair of edges that may form a removal tab. In use the user may grasp an exposed edge of one of the discrete peal strips and apply a force to remove the peel strip.

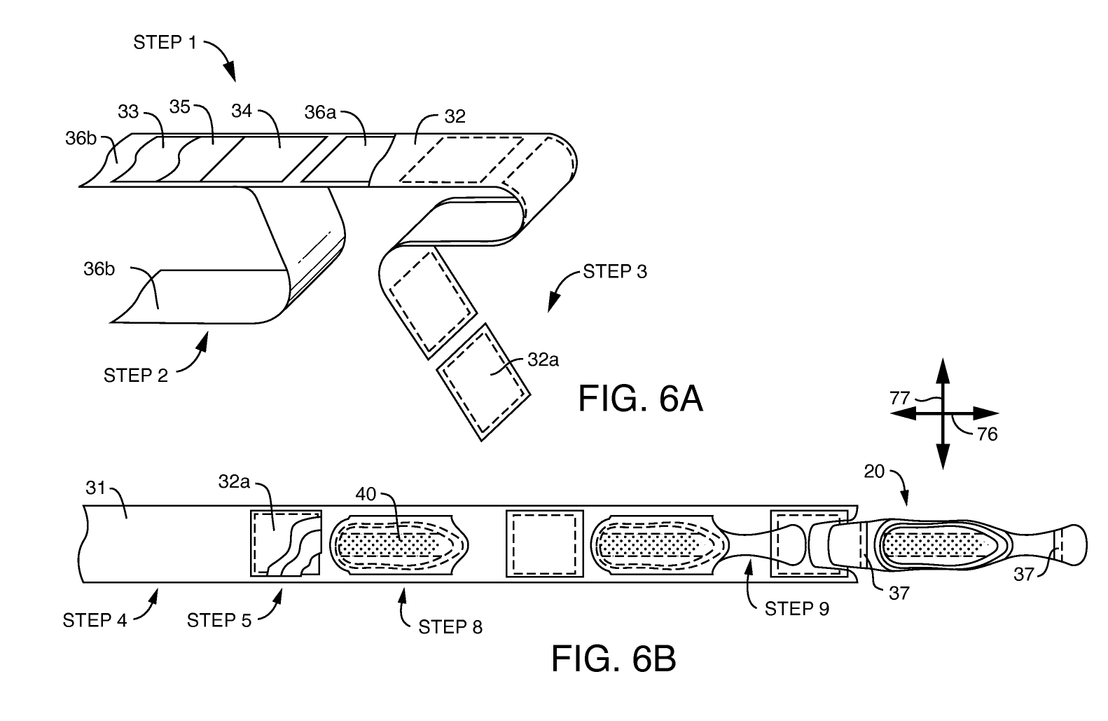

Several methods may be utilized to manufacture the ATL of the present disclosure. For example, as illustrated in FIG. 6, in one embodiment an ATL 32 comprising a peel strip 36, body attachment adhesive 34, and nonwoven carrier 35 may be manufactured by providing a web of nonwoven carrier 35, providing a web of peel strip 36a, applying a body attachment adhesive 34 to the peel strip 36a and curing the adhesive 34, for example by treating the adhesive 34 with ultraviolet light. Once the adhesive 34 is cured to the appropriate level the peel strip adhesive composite is laminated to a nonwoven carrier 35. In a preferred embodiment the body attachment adhesive layer 34 is narrower than the peel strip 36a and may be narrower than nonwoven carrier 35. This results in the formation of a user grasping section or finger tab 37 on the lateral side edges of ATL web 32. In a particularly preferred embodiment the ATL web 32 is made in a multi-wide configuration, slit into individual lanes and wound onto cores.

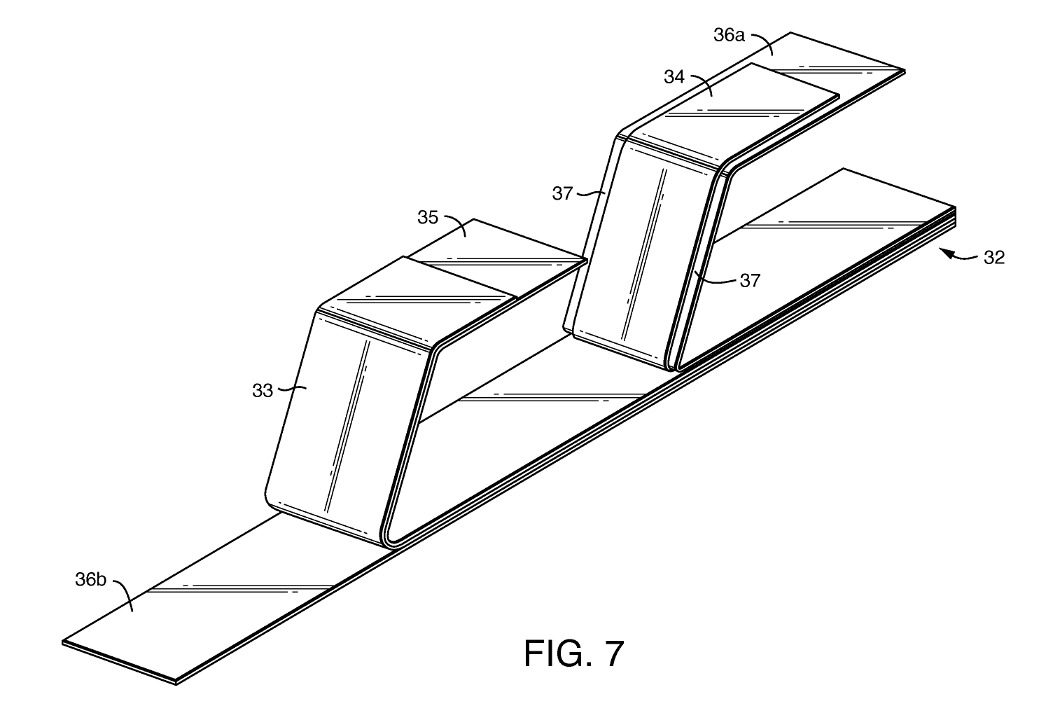

In another embodiment, illustrated in FIG. 7, an ATL 32 comprising a lower peel strip 36b, construction adhesive 33, nonwoven carrier 35, body attachment adhesive 34, and upper peel strip 36a may be manufactured by providing a web of lower peel strip 36b, providing a web of nonwoven carrier 35, applying a construction adhesive 33 to the nonwoven carrier 35, laminating the nonwoven carrier 35 and construction adhesive 33 to the lower peel strip 36b. A web of upper peel strip 36a is provided and a body attachment adhesive 34 is applied to upper peel strip 36a and cured to the appropriate level. The upper peel strip 36a with cured body attachment adhesive 34 is laminated to the nonwoven carrier 35, construction adhesive 33, and lower peel strip 36b laminate.

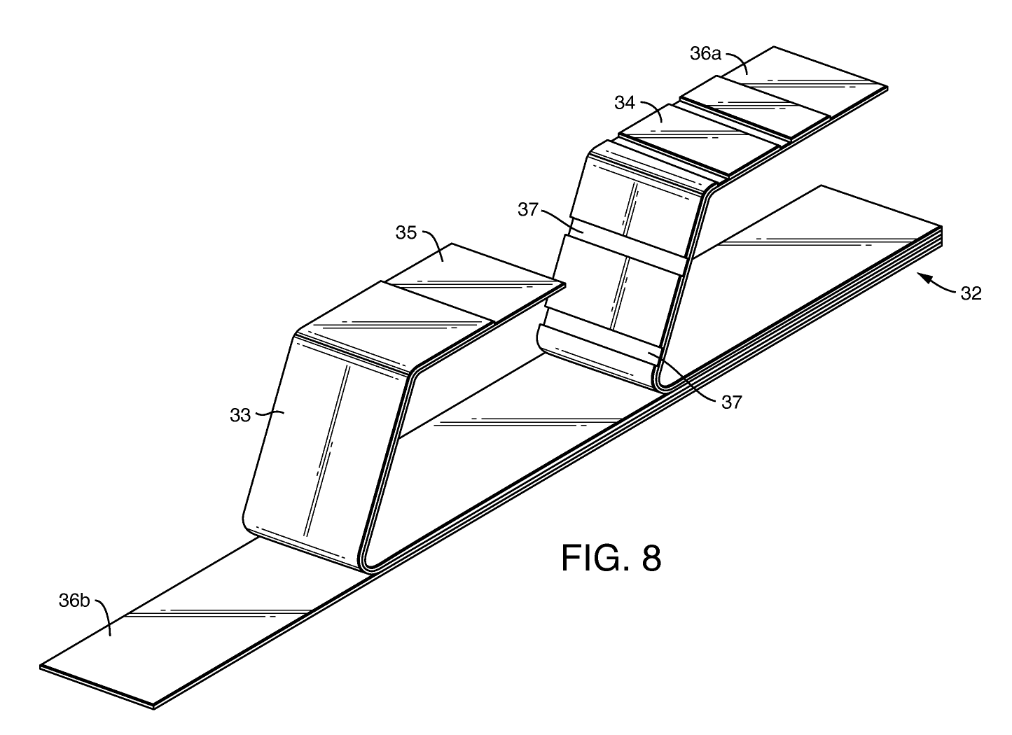

In still another embodiment, illustrated in FIG. 8, an ATL 32 comprising a lower peel strip 36b, construction adhesive 33, nonwoven carrier 35, body attachment adhesive 34, and upper peel strip 36a may be manufactured by providing webs of lower peel strip 36b and nonwoven carrier 35 which are fed into the process, applying construction adhesive 33 to the nonwoven carrier 35 and laminating the nonwoven carrier 35 and construction adhesive 33 composite to the lower peel strip 36b. A web of upper peel strip 36a is provided and discrete patches of body attachment adhesive 34 are applied to upper peel strip 36a and cured. The upper peel strip 36a with cured discrete patches of body attachment adhesive 34 is laminated to the nonwoven carrier 35, construction adhesive 33, and lower peel strip 36b laminate. In this embodiment the discrete patches of body attachment adhesive layer result in the formation of lateral direction user grasping sections 37. ATL 32 lower peel strip 36b and construction adhesive 33 may or may not be needed depending upon the method of making the body adhering absorbent article 20.

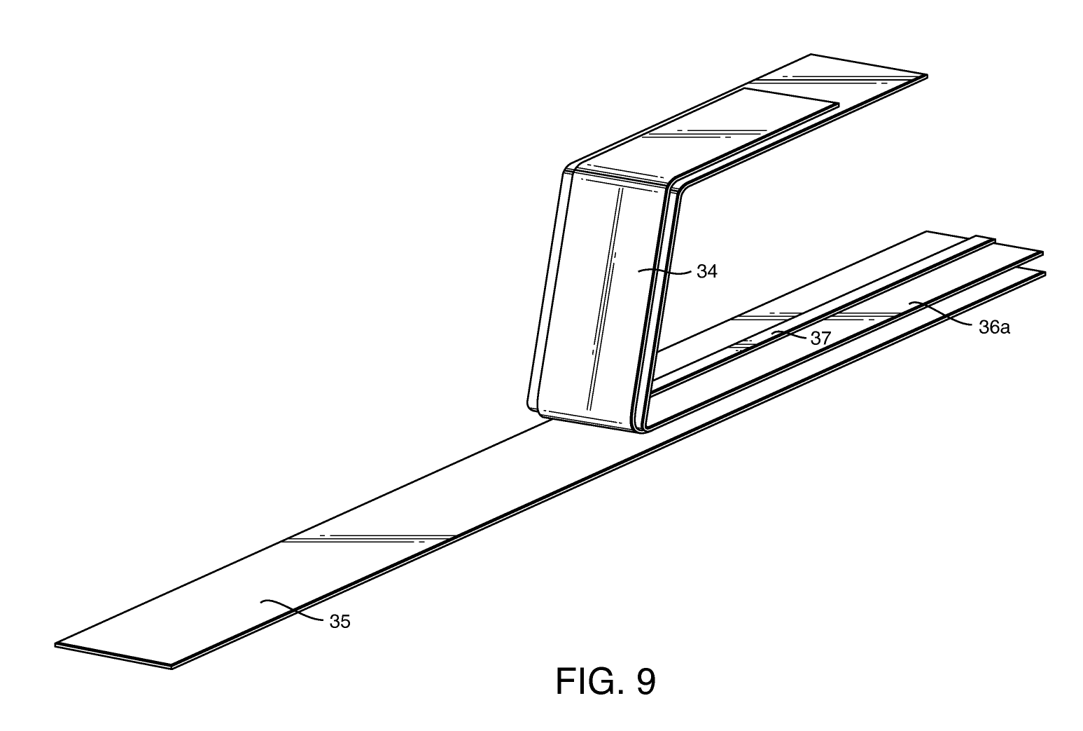

In another embodiment, illustrated in FIG. 9, an ATL 32 comprising a lower peel strip 36b, construction adhesive 33, nonwoven carrier 35, body attachment adhesive 34, and upper peel strip 36a may be manufactured by providing webs of nonwoven carrier 35 and peel strip 36a which are fed into the process. The peel strip 36a is c-folded over onto itself forming user grasping section 37. Body attachment adhesive 34 is applied to the peel strip 36a, cured and laminated to the nonwoven carrier 35, construction adhesive, and lower peel strip 36b laminate.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

We claim:

1. An adhesive transfer layer comprising a body attachment adhesive, a carrier and a first peel strip.

2. The adhesive transfer layer of claim 1 wherein the first peel strip extends beyond the body attachment adhesive to form a finger tab.

3. The adhesive transfer layer of claim 1 wherein the first peel strip comprises two or more discrete components, the discrete components having a pair of adjacent edges.

4. The adhesive transfer layer of claim 1 further comprising a finger tab attached to the first the peel strip.

5. The adhesive transfer layer of claim 1 wherein the first peel strip is folded to form a finger tab.

6. The adhesive transfer layer of claim 1 further comprising a construction adhesive disposed on the carrier.

7. The adhesive transfer layer of claim 6 further comprising a second peel strip disposed on the construction adhesive.

8. The adhesive transfer layer of claim 1 wherein the body attachment adhesive is a UV-curable adhesive.

9. A method of manufacturing an adhesive transfer layer comprising the steps of:
a. providing a web of carrier;
b. providing a web of peel strip;
c. applying a body attachment adhesive either to the peel strip or to the carrier; and
d. attaching the peel strip to the carrier.

10. The method of claim 9 wherein the body attachment adhesive is a UV-curable adhesive and the method further comprises the step of curing the body attachment adhesive.

11. The method of claim 9 wherein the body attachment adhesive layer applied to the peel strip in a pattern such that the adhesive is narrower than the peel strip thereby forming a finger tab.

12. The method of claim 9 wherein the peel strip extends beyond the body attachment adhesive to form a finger tab.

13. A method of manufacturing an adhesive transfer layer comprising the steps of:
a. providing a lower web of peel strip and a web of carrier;
b. applying construction adhesive to the carrier;
c. attaching the carrier to the lower web of peel strip;
d. supplying an upper web of peel strip;
e. applying body attachment adhesive to the upper web of peel strip; and
f. attaching the carrier and the upper web of peel strip.

14. The method of claim 13 wherein the body attachment adhesive is a UV-curable adhesive and the method further comprises the step of curing the body attachment adhesive.

15. The method of claim 13 wherein the width of the body attachment adhesive layer applied to the upper web of peel strip is narrower than the upper web of peel strip.

16. The method of claim 13 wherein the width of the body attachment adhesive layer applied to the upper web of peel strip is narrower than the web of carrier thereby forming a finger tab.

17. A method of manufacturing an adhesive transfer layer comprising the steps of:
a. providing a carrier web and a first peel strip web;
b. c-folding the peel strip to form a user grasping section;
c. applying body attachment adhesive to the peel strip; and
d. attaching the first peel strip to the carrier.

18. The method of claim 17 wherein the body attachment adhesive is a UV-curable adhesive and the method further comprises the step of curing the body attachment adhesive.

19. The method of claim 17 wherein the width of the body attachment adhesive layer applied to the upper web of peel strip is narrower than the upper web of peel strip.

20. The method of claim 17 further comprising the steps of applying a construction adhesive to the carrier and providing a second peel strip web and attaching the second peel strip web to the carrier.
